(19) United States
(12) Patent Application Publication
(10) Pub. No.: US 2011/0162112 A1
(43) Pub. Date: Jun. 30, 2011

(54) INCREASING TIME-EFFICIENCY OF HIGH-THROUGHPUT TRANSFORMATION PROCESSES

(75) Inventors: Yinghong Li, Urbandale, IA (US); Igor C. Oliveira, Johnston, IA (US)

(73) Assignee: Pioneer Hi-Bred International, Inc., Johnston, IA (US)

(21) Appl. No.: 12/976,555

(22) Filed: Dec. 22, 2010

Related U.S. Application Data
(60) Provisional application No. 61/291,674, filed on Dec. 31, 2009.

(51) Int. Cl. C12N 15/82; C12N 15/87; C12N 15/89

(52) U.S. Cl. 800/292; 800/278; 800/293; 800/294

(57) ABSTRACT

The methods provided relate to efficient methods for transforming isolated, immature maize embryos and for producing transgenic maize plantlets. The time required for the production of the transgenic plantlets and subsequent plants is significantly decreased compared to conventional methods. The methods also relate to decreasing the selection time of transgenic events and regenerating a transgenic maize plantlet from transgenic somatic embryos from the events in a plant cell culture vessel that allows for root formation and plantlet elongation in the same plant cell culture vessel.

### CROSS-REFERENCE TO RELATED APPLICATIONS

This application claims the benefit of U.S. Provisional Application 61/291,674, filed Dec. 31, 2009, herein incorporated by reference it its entirety.

### FIELD OF THE INVENTION

This invention is in the field of biotechnology; in particular, this pertains to methods for efficiently transforming immature maize embryos and for producing transgenic maize plantlets.

### BACKGROUND OF THE INVENTION

The development of methods for the introduction of foreign genes into organisms has had a profound impact on the field of agriculture and crop improvement with Agrobacterium-mediated transformation and direct DNA transfer, such as Polyethylene Glycol (PEG)-mediated DNA uptake, electroporation, and biolistics being some of the most widely used methods. These methods have allowed the creation of genetically engineered plants which could not have been obtained by traditional breeding methods. The discovery of novel techniques to transfer genes into plant cells and the development of methods to regenerate plants from these cells or tissue has advanced the field of crop improvement. Despite these advances and the extensive amount of time, money, and energy spent on the production of transgenic plants via Agrobacterium-mediated transformation or direct DNA uptake, many problems remain that are associated with efficient production of transgenic plants. Regeneration of intact plants from transformed tissue is not always an easy task as tissue culture-induced variation, time factors for the recovery of transformants, labor intensive protocols and limitations in regenerating plants from calli exist. There is a need for a transformation protocol which allows for efficient production of transgenic plants.

### SUMMARY OF THE INVENTION

Methods are provided for efficiently transforming immature maize embryos and for producing transgenic maize plantlets. The methods can be used for the incorporation of new traits into cultivated maize plants. The methods comprise obtaining immature embryos from a maize plant and introducing a nucleotide construct into cells from the immature embryos and placing the immature embryo in or on selection medium for no more than 2 rounds of selection. The transgenic callus comprising immature somatic embryos identified during the selection step is cultured in or on a maturation medium to produce mature somatic embryos. The methods additionally include regenerating the mature somatic embryos into transgenic maize plantlets having the polynucleotide of interest using a plant cell culture vessel that allows for root formation and plantlet elongation in the same plant cell culture vessel.

### BRIEF DESCRIPTION OF THE DRAWINGS

The invention can be more fully understood from the following detailed description and the accompanying drawings which form a part of this application.

FIG. 1 shows a schematic showing one embodiment of Agrobacterium-transformation of maize and regeneration using a method of the present invention (on the left) as compared to a conventional method (shown on the right).

FIG. 2 shows a flow chart showing a 6 step Agrobacterium Transformation Protocol of GS3XGF with selection by PAT. Further details are provided in Example 2 herein below.

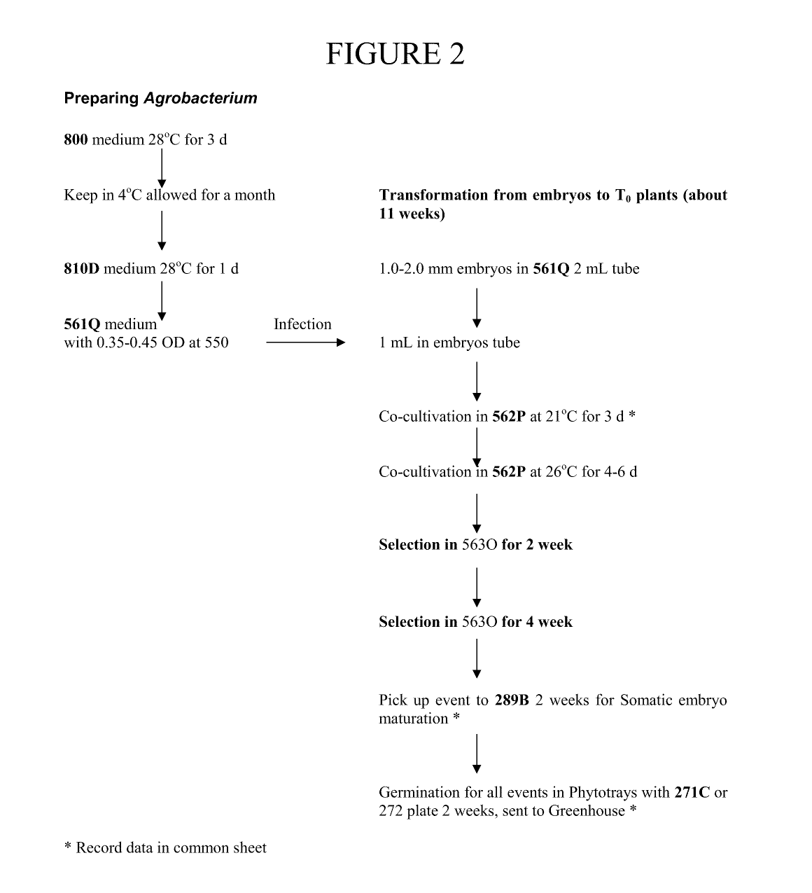

FIG. 3 shows a flow chart showing a 8 step Agrobacterium Transformation Protocol of GS3XGF with selection by PAT. Further details are provided in Example 3 herein below.

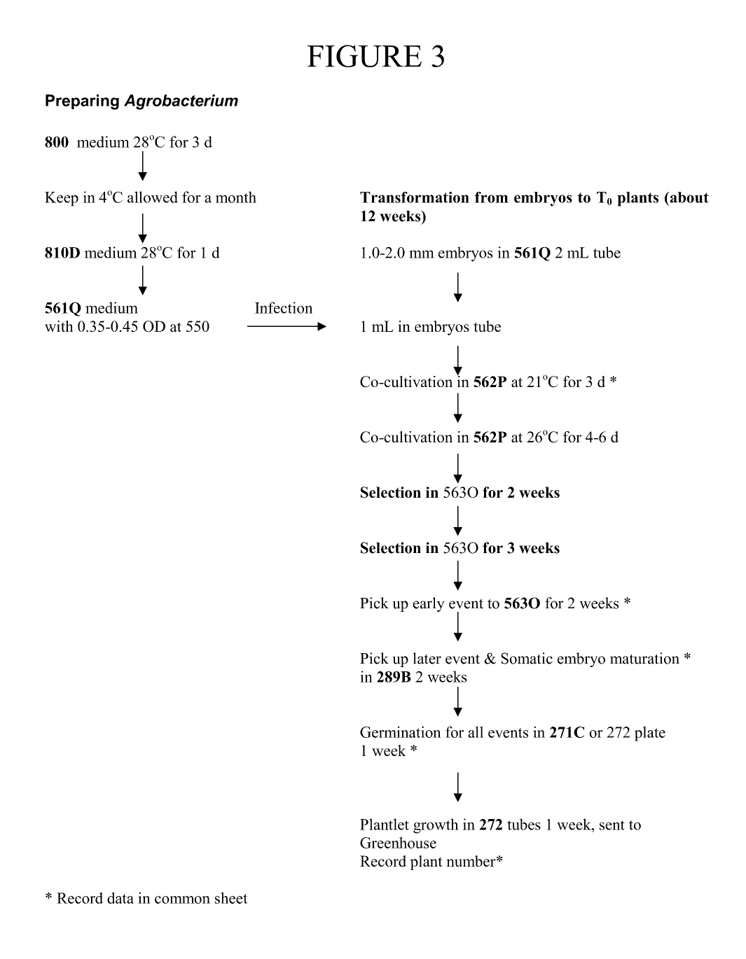

FIG. 4 shows a flow chart showing a 6 step Agrobacterium Transformation Protocol of GS3XGF with selection by GAT. Further details are provided in Example 4 herein below.

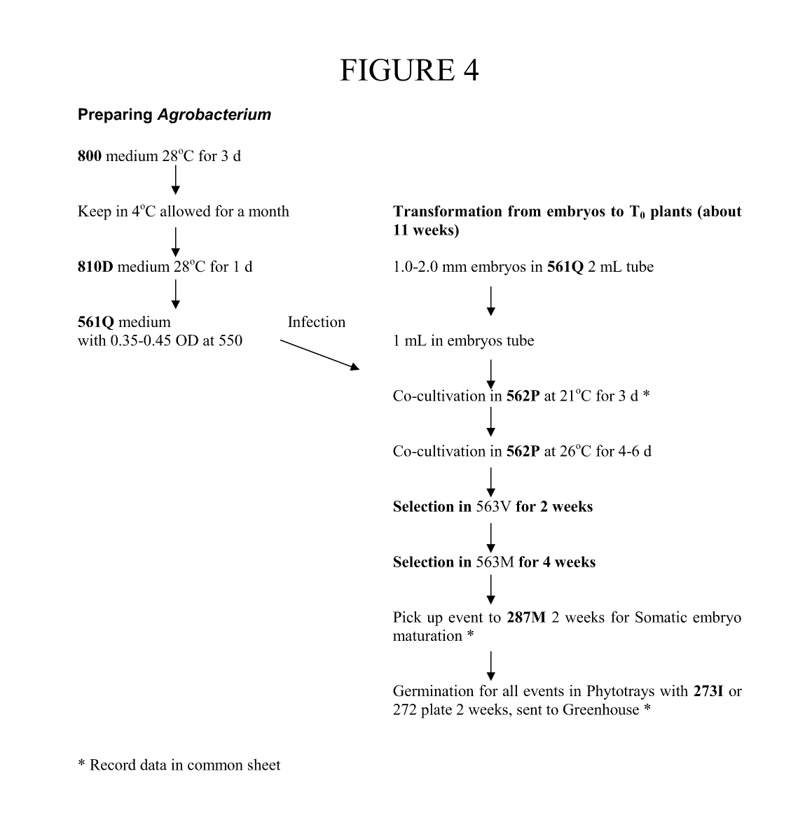

FIG. 5 shows a flow chart showing a 8 step Agrobacterium Transformation Protocol of GS3XGF with selection by GAT. Further details are provided in Example 5 herein below.

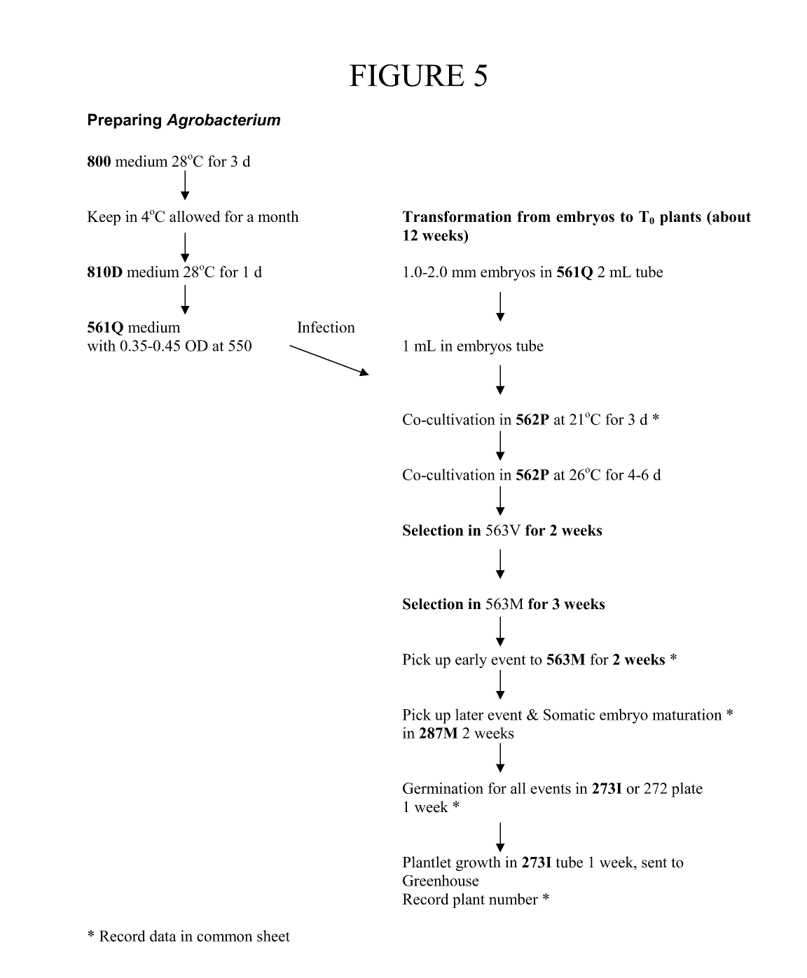

### DETAILED DESCRIPTION OF THE INVENTION

Unless defined otherwise, all technical and scientific terms used herein have the same meaning as commonly understood by one of ordinary skill in the art to which this invention belongs.

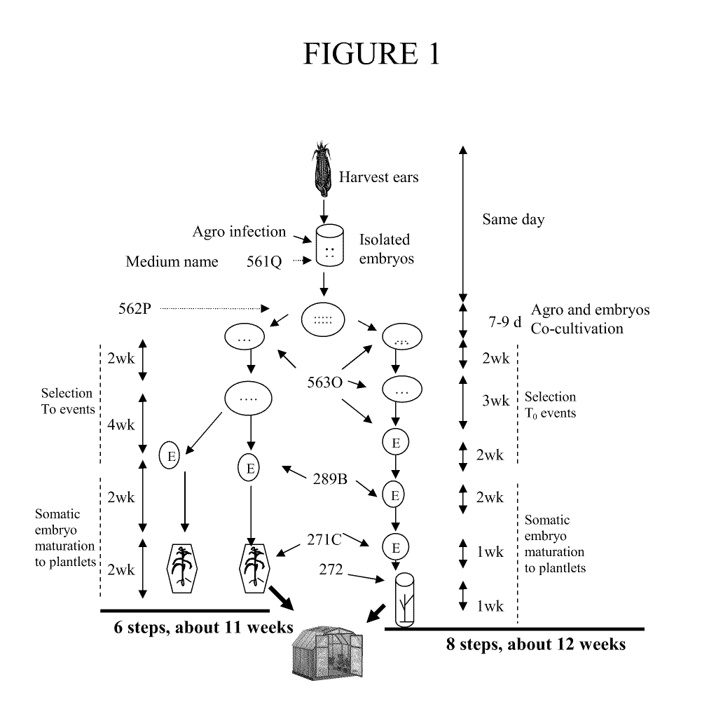

Described herein are efficient methods for maize plant transformation and regeneration. Methods include transforming maize cells and regenerating transgenic maize plantlets. The invention is drawn to methods for introducing nucleotide constructs into cells from maize plants and for producing stably transformed maize plants. The methods find use in efficiently developing new maize cultivars with improved agronomic characteristics. In particular, the methods involve introducing the nucleotide constructs into cells from immature embryos and selecting for transformed cells using one or two rounds of selection. The total period of selection is carried out for about 6 weeks or less, which is at least one week less than other methods. See, for example, FIG. 1, showing an 8 step method to obtain T₀ plants from transformed embryos that undergo 3 rounds of selection for a total of at least 7 weeks.

The transformed cells are proliferated into transgenic callus. The transgenic callus comprising immature somatic embryos are cultured in or on a maturation medium to produce mature somatic embryos. The methods additionally include regenerating the mature somatic embryos into transgenic maize plantlets having the polynucleotide of interest. The regeneration takes place in a plant cell culture vessel that allows for root formation and plantlet elongation in the same vessel. This is in contrast to conventional methods where the plantlet is regenerated in two separate plant cell culture vessels. The initial vessel, such as a petri dish, allows for root formation and the plantlet is transferred to a second vessel, most often a test tube, that provides adequate space for plantlet elongation to occur. A significant advantage of the methods described herein is that, due to the shortened time during the selection step and elimination of a transfer step, the time to regenerate a plantlet or plant is shortened. For example, as shown in FIG. 1, in one aspect, practice of the methods described herein may be used to obtain a T₀ plant from an immature embryo in about 11 weeks or less. This advantageously provides an increased cost- and time-effective route to transform and regenerate plants without compromising the quality of the process or the plants obtained therefrom.

A number of terms used herein are defined and clarified in the following section.

An immature maize embryo is a maize embryo that is physiologically less mature than the dormant embryo that would occur in a typical, viable, mature maize kernel.

An isolated embryo is intended an embryo dissected from the maize caryopsis.

A somatic embryo is embryo initiated and developed from vegetative or non-gametic cells.

Auxin depleted refers to a culture medium that was prepared without the addition of any auxin or auxin-like growth regulator. A medium that is essentially auxin free or auxin depleted may contain other phytohormones or plant growth regulators.

Phytohormone depleted refers to a culture medium that was prepared without the addition of any phytohormone (also referred to as a plant growth regulator). A medium that is phytohormone depleted is auxin depleted.

An effective amount is an amount of an agent, compound or phytohormone that is capable of causing the desired effect on an organism. It is recognized that an effective amount may vary depending on factors, such as, for example, the organism, the target tissue of the organism, the method of administration, temperature, light, relative humidity and the like. Further, it is recognized that an effective amount of a particular agent may be determined by administering a range of amounts of the agent to an organism and then determining which amount or amounts cause the desired effect.

It is to be understood that as used herein the term “transgenic” includes any cell, cell line, callus, tissue, plant part, or plant the genotype of which has been altered by the presence of a heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

As used herein, the term “plant” includes reference to whole plants, plant organs (e.g., leaves, stems, roots, etc.), seeds, plant cells, and progeny of same. Parts of transgenic plants understood to be within the scope of the invention comprise, for example, plant cells, protoplasts, tissues, callus, embryos as well as flowers, stems, fruits, leaves, and roots originating in transgenic plants or their progeny previously transformed with a DNA molecule of interest and therefore consisting at least in part of transgenic cells, are also provided.

As used herein, the term “plant cell” includes, without limitation, seeds, suspension cultures, embryos, meristematic regions, callus tissue, leaves, roots, shoots, gametophytes, sporophytes, pollen, and microspores. The class of plants that can be used in the methods of the invention is generally as broad as the class of higher plants amenable to transformation techniques, including monocotyledonous plants, in particular maize.

“Transformation” refers to the transfer of a nucleic acid fragment into the genome of a host organism, resulting in genetically stable inheritance. Host organisms containing the transformed nucleic acid fragments are referred to as “transgenic” organisms. Examples of methods of plant transformation include Agrobacterium-mediated transformation (De Blaere et al. (1987) Meth. Enzymol. 143:277) and particle-accelerated or “gene gun” transformation technology (Klein et al. (1987) Nature (London) 327:70-73; U.S. Pat. No. 4,945,050, incorporated herein by reference). Additional transformation methods are disclosed below.

A transgenic “event” is produced by transformation of plant cells with a heterologous DNA construct(s), including a nucleic acid expression cassette that comprises a transgene of interest, the regeneration of a population of plants resulting from the insertion of the transgene into the genome of the plant, and selection of a particular plant characterized by insertion into a particular genome location. An event is characterized phenotypically by the expression of the transgene. At the genetic level, an event is part of the genetic makeup of a plant. The term “event” also refers to progeny produced by a sexual outcross between the transformant and another variety that include the heterologous DNA. Even after repeated back-crossing to a recurrent parent, the inserted DNA and flanking DNA from the transformed parent is present in the progeny of the cross at the same chromosomal location. The term “event” also refers to DNA from the original transformant comprising the inserted DNA and flanking sequence immediately adjacent to the inserted DNA that would be expected to be transferred to a progeny that receives inserted DNA including the transgene of interest as the result of a sexual cross of one parental line that includes the inserted DNA (e.g., the original transformant and progeny resulting from selfing) and a parental line that does not contain the inserted DNA.

Pre-cultured embryos are embryos cultured prior to bombardment on a medium which promotes the production of embryogenic tissue and precedes the conditioning of the embryo in preparation for transformation.

Pre-cultured embryos of maize are typically cultured for a period to produce an embryogenic response prior to particle bombardment. The tissue derived from the embryogenic response provides the target cells for transformation. Conditions during this period of pre-bombardment culture generally include a plant growth regulator and a period of time generally from one to seven days or more. The particular conditions depend on the culture medium formulation, genotype, and general health of the donor plant.

Efficient methods for obtaining stably transformed maize plants are provided. The methods can involve the use of a transformation medium comprising a high concentration of an osmoticum. The osmoticum can include compounds that are known to be metabolized by plants, and/or compounds that are not known to be metabolized by plants. Osmoticum that are known to be metabolized by plants include but are not limited to osmoticum such as, for example, sucrose, glucose, fructose and maltose, which are routinely used as a carbon source in plant culture media (Vain et al. (1993) Plant Cell Rep 12:84-88) and immature maize embryos (Brettschneider et al. (1997) Theor Appl Genet. 94:737-748, Pareddy et al. (1997) Maydica 42:143-154; Dunder et al. (1995) In: Gene Transfer to Plants (Potrykus and Spangenberg, eds.) Springer-Verlag, NY, pp. 127-138).

A high concentration of an osmoticum is a concentration that is higher than that typically used when the osmoticum is intended solely as a carbon source. The concentration may be any amount over the standard concentration used in the medium, including but not limited to 0.1%, 0.5%, 1%, 5%, 10%, 20%, 50%, 100%, 200%, or 500% or more higher than the standard concentration. The concentration can be denoted in any units including but not limited to weight/volume (w/v), volume/volume (v/v), molarity, molality, or g/liter. For example, sucrose is routinely used at a concentration of about 3% (w/v) as a carbon source in plant culture media. A high concentration of sucrose in a medium is a concentration that exceeds 3% (w/v). For other osmoticum, including those known to be metabolized by plants and those that are not known to be metabolized by plants, a “high concentration” is a concentration that generally exceeds the molar concentration of sucrose in a medium comprising 3% (w/v) sucrose. The osmoticum may be 8%, 12%, 19% or 30% w/v. Optionally, the osmoticum may be 12-19%.

The method encompasses the use of both solid and liquid plant culture media. Those of ordinary skill in the art recognize that the preparation of solid plant culture media typically involves dissolving or suspending the various media components in a solution comprising water. It is recognized that the concentrations of components of such solid media referred herein are the concentrations of the components in the aqueous solution prior to solidification or gelling.

The methods generally employ immature maize embryos. Such embryos are generally isolated from a maize ear that was pollinated less than about 16 days before use, embryos can be pollinated between about 6 and about 16 days before use, embryos are most frequently pollinated between about 9 and about 12 days before use. Generally, such embryos are between about 1.5 mm and 1.8 mm in length measured from the coleoptilar top to end to the coleorhizal end. Sizing of embryo for explant and transformation is best accomplished by developmental staging rather than by absolute size. Immature embryos are initially translucent. It is when the entire embryo, axis and scutellum, first become opaque, that they reach the proper developmental stage for use in the process. Immature embryos are generally cultured as soon after they become opaque as possible. Size of embryo (length) is roughly correlated with opacity, but both genotype and environment have dramatic altering effects on embryos size.

Such ears may be obtained from any source, including field, greenhouse and/or growth-chamber grown maize plants. Typically, the ear is harvested from the maize plant before isolation of the embryos therein, and is subsequently sterilized or otherwise treated to reduce undesired biological contamination, particularly microbial contamination. Methods are known in the art for reducing or eliminating microbial contamination from live plant tissues, such as maize ears, including, but not limited to, contacting the ear, typically after removal of the husk, with an aqueous solution comprising household laundry bleach.

The methods involve the use of isolated, immature embryos. In one method, the immature embryos are isolated from ears that were harvested in the same 24-hour period as the embryo isolation. However, the methods also encompass the use of ears that are stored for a period of time before embryo isolation. Any method of storing ears may be employed. It is recognized, however, that selected methods of ear storage conditions will substantially preserve the viability of the immature embryos therein. The age of an embryo is determined as the interval of time from pollination of the ear to isolation of the embryo therefrom.

The immature embryos may be obtained from a maize plant by any method known in the art. Typically, the embryos will be isolated from a de-husked ear by excising with a sharp-bladed instrument such as, for example, a scalpel, knife or other sharp instrument. Upon isolation from an ear, the immature embryos are typically contacted with transformation medium. However, it is recognized that the immature embryos may be contacted with one or more alternative media before contacting the transformation medium. It is further recognized that such alternative media are media that are not known to promote the formation of embryogenic maize callus and are preferably auxin-depleted or phytohormone-depleted media. Such alternative media may optionally comprise a high concentration of an osmoticum. Further it is recognized that contacting comprises both direct contact of an immature embryo with a medium and indirect contact such as, for example, an immature embryo placed on one side of a filter paper that has its opposite side in contact with the medium.

After contacting an isolated, immature embryo with transformation medium, a nucleotide construct may be introduced into a cell of the embryo immediately thereafter or following a period of time, usually not more than about 24 hours after isolation of the immature embryo.

The type of transformation method is not critical to the methods; various methods of transformation are currently available. As newer methods are available to transform host cells they may be directly applied. Accordingly, a wide variety of methods have been developed to insert a DNA sequence into the genome of a host cell to obtain the transcription and/or translation of the sequence. Thus, any method that provides for efficient transformation/transfection may be employed.

Methods for transforming various host cells are disclosed in Klein et al. (1992) Bio/Technol 10:286-291. Techniques for transforming a wide variety of higher plant species are well known and described in the technical, scientific, and patent literature. See, for example, Weising et al. (1988) Ann Rev Genet. 22:421-477. For example, the DNA construct may be introduced directly into the genomic DNA of the plant cell using techniques such as electroporation, PEG-induced transfection, particle bombardment, silicon fiber delivery, or microinjection. See, e.g., Tomes et al., Direct DNA Transfer into Intact Plant Cells Via Microprojectile Bombardment. pp. 197-213 in Plant Cell, Tissue and Organ Culture, Fundamental Methods. eds. Gamborg and Phillips. Springer-Verlag Berlin Heidelberg New York, 1995. The introduction of DNA constructs using polyethylene glycol precipitation is described in Paszkowski et al. (1984) EMBO J. 3:2717-2722. Electroporation techniques are described in Fromm et al. (1985) Proc Natl Acad Sci USA 82:5824. Ballistic transformation techniques are described in Klein et al. (1987) Nature 327:70-73. The methods could also involve microprojectile bombardment to introduce nucleotide constructs into the cells of isolated, immature maize embryos. In particular, microprojectile bombardment may be conducted using a high pressure gas delivery system such as, for example, the invention described in U.S. Pat. No. 5,204,253, for which an embodiment known as Biolistic PDS-1000/He System is available commercially, or using any other device known in the art which is capable of delivering to a cell a nucleotide construct on or in microprojectiles.

If desired, the immature embryo may be oriented on the transformation medium for introduction of the nucleotide construct. For introduction by microprojectile bombardment, the immature embryos may be orientated to optimize entry of the nucleotide-construct-coated microprojectiles into a particular region of the immature embryo. Typically for microprojectile bombardment, the immature embryos are oriented with the scutellum of the immature embryos directly facing the expected path of the nucleotide-construct-coated microprojectiles. It is contemplated that the medium be solid, semi-solid or a solid surface floating on top of a liquid or semi-liquid surface (e.g., filter paper on liquid).

Alternatively, the DNA constructs may be combined with suitable T-DNA flanking regions and introduced into a bacterial vector for plant transformation, such as a Rhizobiaceae vector, including but not limited to Agrobacterium rhizogenes or Agrobacterium tumefaciens host vectors. For example, the virulence functions of the Agrobacterium tumefaciens host will direct the insertion of the construct and adjacent marker into the plant cell DNA when the cell is infected by the bacteria. Agrobacterium tumefaciens-meditated transformation techniques are well described in the scientific literature. See, for example Horsch et al. (1984) Science 233:496-498, and Fraley et al. (1983) Proc Natl Acad Sci USA 80:4803. For instance, Agrobacterium transformation of maize is described in U.S. Pat. No. 5,981,840. Agrobacterium transformation of monocots is found in U.S. Pat. No. 5,591,616. Agrobacterium transformation of soybeans is described in U.S. Pat. No. 5,563,055.

Other methods of transformation include: Agrobacterium rhizogenes-induced transformation (see, e.g., Lichtenstein and Fuller In: Genetic Engineering, vol. 6, P W J Rigby, Ed., London, Academic Press, 1987; and Lichtenstein and Draper, In: DNA Cloning, Vol. II, D. M. Glover, Ed., Oxford, IRI Press, 1985), Application PCT/US87/02512 (WO 88/02405 published Apr. 7, 1988) describes the use of A. rhizogenes strain A4 and its Ri plasmid along with A. tumefaciens vectors pARC8 or pARC16; liposome-induced DNA uptake (see, e.g., Freeman et al. (1984) Plant Cell Physiol 25:1353); and vortexing methods (see, e.g., Kindle (1990) Proc Natl Acad Sci USA 87:1228).

After the introduction of the nucleotide construct, the immature embryos may be transferred to an identification or selection medium, a maturation medium, a regeneration medium, or a medium that is for both identification/selection and maturation/regeneration. Such media may comprise an auxin, for example 2,4-dichlorophenoxyacetate (2,4-D). Alternatively, an auxin can be added to a plate containing an auxin-depleted medium. The transfer to another medium or the addition of auxin to the medium may occur immediately following the introduction of the nucleotide construct or, if desired, after a period of time. Typically within about one week or less after the introduction of the nucleotide construct, the immature embryos are transferred from the transformation medium to another medium, or auxin is added to the transformation medium. Usually the embryos are transferred to another medium, or auxin is added to the transformation medium, within about 2 to about 3 days after introduction of the nucleotide construct. Generally, the medium that the immature embryos are transferred to after introduction of the nucleotide construct will depend on the method by which the nucleotide construct was introduced into cells of the immature embryos, the nucleotide construct and the desired outcome. The medium used may additionally comprise other components such as, for example, antibiotics.

The transformed cells may be identified or selected and, if desired, regenerated into transformed plants. The transformed cells from immature embryos may be selected, identified and regenerated into transgenic maize plantlets and plants. Any techniques known in the art for identifying transformed cells may be used in conjunction with the methods described herein. Identification methods may involve utilizing a marker gene, such as YFP, CPF, RFP, GFP, or any other fluorescent marker, or a cell cycle gene, such as CKI, or cyclin D. Methods for using GFP and cell cycle genes are found in U.S. Pat. Nos. 6,300,543, 6,518,487, and 7,256,280, herein incorporated by reference.

Selection methods typically involve placing the immature embryos, or parts thereof, in or on a medium that contains a selective agent and allows proliferation of the transformed cells to produce transgenic callus containing immature somatic embryos. If, for example, the nucleotide construct comprises a selectable marker gene for herbicide resistance that is operably linked to a promoter that drives expression in a plant cell, then selection of the transformed cells may be achieved by adding an effective amount of the herbicide to the medium to inhibit the growth of or kill non-transformed cells. Such selectable marker genes and methods of use are well known in the art.

When the immature embryos are transformed using Agrobacterium, following the co-cultivation step, or following the resting step where it is used, the selection medium may include an antibiotic to inhibit growth of the Agrobacterium. Generally, any of the media known in the art suitable for the culture of the plant cell of interest can be used in the selection step. During selection, the transformed cells/tissues are typically cultured until callus formation, i.e. a transformation event, is observed. The transformed cells/tissues are selected on selection medium for about 6 weeks or less. During that period of time, a second round of selection or subculturing of transformed cells in or on selection medium, e.g. fresh or renewed medium, may be desirable to provide an ample supply of nutrients to promote growth of the transformed cells into transgenic callus. Furthermore, subculturing of the embryos can facilitate the identification and isolation of transformed cells forming a transgenic callus, i.e. a putative transformation event. Accordingly, in some cases, the methods include selecting for transformed cells for a total of about 6 weeks or less, including a subculture step. The cells may be subcultured once after the 2, 3 or 4 weeks of initial exposure to the selection medium for further selection for another 2, 3 or 4 weeks. This is contrary to teachings or published protocols that selection of transformed cells/tissue is best performed for at least 7 or more weeks, including 2 to 3 subculturings at 2-3 week intervals, to effectively kill any persistent Agrobacterium present in the culture and to prevent crowding of events thereby allowing for the isolation of single proliferated events. (Zuo-yu Zhao, Weining Gu, Tishu Cai, Laura Tagliani, David Hondred, Diane Bond, Sheryl Schroeder, Marjorie Rudert and Dottie Pierce, High throughput genetic transformation mediated by Agrobacterium tumefaciens in maize, Molecular Breeding 8: 323-333, 2001.) See, also, FIG. 1. Surviving transgenic callus comprising immature somatic embryos is transferred to or contacted with a maturation medium. Generally, any of the media known in the art suitable for the culture of the plant cell of interest can be used in the maturation step. Typically the maturation media includes ABA (plant hormone contained in maturation medium 289B). After exposure to maturation medium immature somatic embryos give rise to mature somatic embryos. Mature somatic embryos are usually obtained in about 2-4 weeks.

Methods and media employed in the regeneration of transformed maize plants from transformed cells of immature embryos are known in the art. Generally, such methods comprise contacting the mature somatic embryo with a regeneration medium. Typically, the mature somatic embryos which are regenerated from tissue derived from each unique event are then cultured in a petri dish on an appropriate medium in a light cycle until shoots and roots develop for approximately one week. Individual small plantlets are then selected and their roots trimmed using aseptic technique so that each plantlet can be placed inside a test tube without its roots touching the outside of the test tube to prevent contamination. Each tube containing the plantlet contains regenerating medium to allow the plantlet to grow and develop longer roots for approximately another week at which time each plantlet is transplanted to a soil mixture in pots in the greenhouse.

Advantageously, using the methods described herein, the time required for the transformation process can be shortened during the selection step. The transformation process can be simplified further if during regeneration the mature somatic embryos are regenerated into plantlets in a plant culture vessel that allows for root formation and plantlet elongation in the same plant cell culture vessel. Doing so eliminates the subsequent transfer of the plantlet from a petri dish to a test tube to a pot. Performing the conventional transfer steps during regeneration are generally inconvenient since the steps involve twice the effort, which is manageable for small scale transformation but for relatively high throughput transformation, a two-step transfer process is much more labor and time-intensive than desirable or necessary. Further, not only does the elimination of one transfer step equate to less technician time, it also means less-handling, which translates into a decreased opportunity for contamination or physical damage to the plantlet to occur. This advantageously provides an increased cost- and time-effective route to transform and regenerate plants without compromising the quality of the process or the plants obtained therefrom. For example, the number of vectors or constructs transformed into embryos over a given time period using the methods described herein increased by as much as 2-fold without sacrificing the number or quality of the plants produced. Moreover, the man-hours expended using a 6-step, approximately 11 week process to obtain a T₀ plant as compared to an 8-step, approximately 12 week process may be significantly decreased by as much as 5%, 10%, 11%, 12%, 13%, 14%, 15%, 16%, 17%, 18%, 19%, 20% or greater.

Table 1 shows a comparison between the “long” (approximately 12 week) and “short” (approximately 11 week) transformation protocols. In particular, note the doubling in transformation capacity achieved. Transformation efficiency is measured by infecting one ear of maize with vectors to obtain 8-10 T₀ plants/vector to send to a greenhouse. The methods compared are use of an 8 step, approximately 12 week transformation method, versus a 6 step, approximately 11 week transformation method.

TABLE 1
Comparison Between Long and Short Transformation Protocols

| | | No. of Vectors | No. of T₀ |
|---|---|---|---|
| | Relative No. | Producing | Plants per |
| | of Vectors | 8-10 T₀ | Vector |
| Process Description | Transformed | Plants (%) | (Average) |
| 8 step, ~12 week process* | 1X | 89.1 | 9.1 |
| 6 step, ~11 week process | 2X | 91.7 | 9.2 |
| (pilot exp)** | | | |
| 6 step, ~11 week process | N/A | 95.3 | 9.6 |
| (routine run)*** | | | |

*Typical outcome of a “regular” (i.e., long, ~12 week) transformation process.
**Initial pilot experiment to test the short transformation protocol. Here one can see that the efficiencies of the short process (event generation/vector) were indistinguishable from that of the long transformation process at 2X the number of transformed vectors;
***Data on the efficiencies of the short protocol was collected over the period of a year to show the sustainability and consistency of the process.

Use of a 6 step, about 11 week transformation method described herein allows the number of constructs transformed into ears to obtain 8-10 T₀ plants to be increased by at least 30%, 40%, 50% or 60% or greater as compared to an using an 8 step, about 12 week transformation method. Thus, in one aspect, shortening selection time of transformed embryos while omitting a round of selection and transfer of plantlets to another vessel shortens the length of time to produce plants while simultaneously doubling the process through-put.

Accordingly, the methods include regenerating the mature somatic embryos into plantlets in any suitable cell culture vessel that allows for the formation of roots and elongation of plantlets in the same container prior to the plantlet being placed in soil in a pot. Suitable cell culture vessels include but are not limited to phytotray plant cell culture vessels (Sigma, St. Louis, Mo.), any sterile vessels with space for plantlet elongation, and the like. Such vessels exclude test tubes or shallow dishes such as petri dishes that do not provide adequate depth for plantlet elongation. A further advantage from using the same plant cell culture vessel is that it allows for multiple plantlets grow in a single vessel, which is ergonomically easier and faster than conventional methods that require aseptic techniques, e.g. sterile instruments to cut the roots of a plantlet, to place an individual plantlet within the narrow confines of the test tube. Using the same plant cell culture vessel allows the greenhouse technician to use fingers to separate out the desired individual plantlet for potting and subsequently growing it to maturity.

The methods do not depend on a particular nucleotide construct. Any nucleotide construct that may be introduced into a plant cell may be employed in the methods. Nucleotide constructs comprise at least one nucleotide sequence of interest, optionally the nucleotide sequence of interest is operably linked to a promoter that drives expression in a plant cell. The nucleotide constructs may also comprise identification or selectable marker gene constructs in addition to the nucleotide sequence of interest.

Selectable marker genes may be utilized for the selection of transformed cells or tissues. Selectable marker genes include genes encoding antibiotic resistance, such as nptII which encodes neomycin phosphotransferase II (NEO), hpt which encodes hygromycin phosphotransferase (HPT), and the moncot-optimized cyanamide hydratase gene (moCAH) (see U.S. Pat. No. 6,096,947) as well as genes conferring resistance to herbicidal compounds, such as glufosinate ammonium, bromoxynil, imidazolinones, and 2,4-dichlorophenoxyacetate (2,4-D). See generally, Yarranton (1992) Curr Opin Biotech 3:506-511; Christopherson et al. (1992) Proc Natl Acad Sci USA 89:6314-6318; Yao et al. (1992) Cell 71:63-72; Reznikoff (1992) Mol Microbiol 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc Natl Acad Sci USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc Natl Acad Sci USA 90:1917-1921; Labow et al. (1990) Mol Cell Biol 10:3343-3356; Zambretti et al. (1992) Proc Natl Acad Sci USA 89:3952-3956; Baim et al. (1991) Proc Natl Acad Sci USA 88:5072-5076; Wyborski et al. (1991) Nucl Acids Res 19:4647-4653; Hillenand-Wissman (1989) Topics Mol Struc Biol. 10:143-162; Degenkolb et al. (1991) Antimicrob Agents Chemother 35:1591-1595; Kleinschnidt et al. (1988) Biochemistry 27:1094-1104; Bonin (1993) Ph.D. Thesis, University of Heidelberg; Gossen et al. (1992) Proc Natl Acad Sci USA 89:5547-5551; Oliva et al. (1992) Antimicrob Agents Chemother 36:913-919; Hlavka et al. (1985) Handbook of Experimental Pharmacology, Vol. 78 (Springer-Verlag, Berlin); Gill et al. (1988) Nature 334:721-724; all of which are herein incorporated by reference. The above list of selectable marker genes is not meant to be limiting. Any selectable marker gene can be used in the present invention. Other marker genes such as GFP (WO97/41228) may also be utilized.

Likewise, the methods of the invention do not depend on immature maize embryos of a particular genotype. The methods of the present invention may be used with immature maize embryos of any maize genotype including immature embryos from both hybrids and inbreds. Examples of maize genotypes include, but are not limited to, Hi-II and hybrids of a cross between Hi-II and a second genotype such as, for example, PHN46, PHTE4, PHAA0, PHP18, PH05F, PH09B, PHP02, PHJ90, PH24E, PHT05, ASKC27 and PH21T. Examples of elite maize genotypes include but are not limited to PH179P, PH179R, PHP38, PH17P7, PH17T8, PH182Y, PH18F6, PHAPH, PHAC4, PH12K5, PH12SG, PH12SK, PH17T7, PH6PV, PH705, PH7CH, PHAKC, PHAPH, PHCER, PHE0N, PHE4N, PHE67, PHY71, PHEJW, PHEKJ, PHEKN, PHGJ4, PHGMG, PHH4V, PHH5G, PHH7E, PHHC6, PHHEB, PHHJN, PHR1J, PH12P5, PHDTD, PHTMM1, PHW0N, PH6WA, PH726, PHP02, PH51H, PHEDR, PHEWB, PH581, PH8JR, PHAJE, PHCJP, PHR03, PHHHN, PHN46, PH1CA, PH4CN, and PHH9H. Elite inbreds are typically inbred maize genotypes that are used to produce commercial hybrid maize lines.

The methods involve producing a stably transformed maize plant. Such a transformed maize plant is a fertile maize plant that is capable of producing at least one transformed progeny. The methods provide a way to identifying viable immature embryos comprising a transformed maize cell having the polynucleotide of interest.

The methods involve the use of plant culture media. Any plant culture medium known in the art may be employed in the methods including, but not limited to, a transformation support medium, identification or selection medium, a maturation medium, and a regeneration medium. Typically, such media comprise water, a basal salt mixture and a carbon source, and may additionally comprise one or more other components known in the art, including but not limited to, vitamins, co-factors, myo-inositol, selection agents, charcoal, amino acids, silver nitrate and phytohormones. If a solid plant culture medium is desired, then the medium additionally comprises a gelling agent such as, for example, gelrite, agar or agarose.

For example, transformation medium 560Y comprises 4.0 g/L N6 basal salts (SIGMA C-1416), 1.0 mL/L Eriksson's Vitamin Mix (1000X SIGMA-1511), 0.5 mg/L thiamine HCl, 120.0 g/L sucrose, 1.0 mg/L 2,4-D, and 2.88 g/L L-proline (brought to volume with D-I H₂0 following adjustment to pH 5.8 with KOH); 2.0 g/L Gelrite (added after bringing to volume with D-I H₂0); and 8.5 mg/L silver nitrate (added after sterilizing the medium and cooling to room temperature). Selection medium 560R comprises 4.0 g/L N6 basal salts (SIGMA C-1416), 1.0 mL/L Eriksson's Vitamin Mix (1000X SIGMA-1511), 0.5 mg/L thiamine HCl, 30.0 g/L sucrose, and 2.0 mg/L 2,4-D (brought to volume with D-I H₂0 following adjustment to pH 5.8 with KOH); 3.0 g/L Gelrite (added after bringing to volume with D-I H₂0); and 0.85 mg/L silver nitrate and 3.0 mg/L bialaphos (both added after sterilizing the medium and cooling to room temperature).

Plant regeneration medium 288J comprises 4.3 g/L MS salts (GIBCO 11117-074), 5.0 mL/L MS vitamins stock solution (0.100 g nicotinic acid, 0.02 g/L thiamine HCL, 0.10 g/L pyridoxine HCL, and 0.40 g/L glycine brought to volume with polished D-I H₂0) (Murashige and Skoog (1962) Physiol. Plant. 15:473), 100 mg/L myo-inositol, 0.5 mg/L zeatin, 60 g/L sucrose, and 1.0 mL/L of 0.1 mM abscisic acid (brought to volume with polished D-I H₂0 after adjusting to pH 5.6); 3.0 g/L Gelrite (added after bringing to volume with D-I H₂0); and 1.0 mg/L indoleacetic acid and 3.0 mg/L bialaphos (added after sterilizing the medium and cooling to 60° C.). Phytohormone-depleted medium 272V comprises 4.3 g/L MS salts (GIBCO 11117-074), 5.0 mL/L MS vitamins stock solution (0.100 g/L nicotinic acid, 0.02 g/L thiamine HCL, 0.10 g/L pyridoxine HCL, and 0.40 g/L glycine brought to volume with polished D-I H₂0), 0.1 g/L myo-inositol, and 40.0 g/L sucrose (brought to volume with polished D-I H₂0 after adjusting pH to 5.6); and 6 g/L bacto-agar (added after bringing to volume with polished D-I H₂0), sterilized and cooled to 60° C.

The methods optionally use phytohormones and/or plant growth regulators such as, for example, auxins, cytokinins, gibberellins and ethylene. The phytohormones may include, but are not limited to, both free and conjugated forms of naturally occurring phytohormones or plant growth regulators. Additionally, the phytohormones encompass synthetic analogues and precursors of such naturally occurring phytohormones and synthetic plant growth regulators.

Naturally occurring and synthetic analogues of auxins and auxin-like growth regulators include, but are not limited to, indoleacetic acid (IAA), 3-indolebutyric acid (IBA), α-napthaleneacetic acid (NAA), 2,4-dichlorophenoxyacetic acid (2,4-D), 4-(2,4-dichlorophenoxy)butyric acid, 2,4,5-trichlorophenoxyacetic acid (2,4,5-T), 3-amino-2,5-dichlorobenzoic acid (chloramben), (4-chloro-2-methylphenoxy)acetic acid (MCPA), 4-(4-chloro-2-methylphenoxy)butanoic acid (MCPB), mecoprop, dicloprop, quinclorac, picloram, triclopyr, clopyralid, fluoroxypyr, dicamba and combinations thereof. It is recognized that such combinations can be comprised of any possible combination of two or more molecules selected from the group consisting of naturally occurring auxins, synthetic analogues of auxins, and auxin-like growth regulators. By “auxin-like growth regulator” is intended a compound that is not considered an auxin but possesses at least one biological activity that is the substantially the same as that of a naturally occurring auxin.

Examples of phytohormones include naturally occurring compounds, synthetic analogues of cytokinins, and cytokinin-like growth regulators including, but not limited to kinetin, zeatin, zeatin riboside, zeatin riboside phosphate, dihydrozeatin, isopentyl adenine 6-benzyladenine and combinations thereof. It is recognized that such combinations can be comprised of any possible combination of two or more molecules selected from the group consisting naturally occurring cytokinins, synthetic analogues of cytokinins and cytokinin-like growth regulators. By “cytokinin-like growth regulator” is intended a compound that is not considered a cytokinin but possesses at least one biological activity that is the substantially the same as that of a naturally occurring cytokinin.

A nucleotide construct includes any polynucleotide molecule that has been isolated and/or modified as compared to its native source, it is not limited to nucleotide constructs comprising DNA. Those of ordinary skill in the art will recognize that nucleotide constructs, particularly polynucleotides and oligonucleotides, comprised of ribonucleotides and combinations of ribonucleotides and deoxyribonucleotides may also be employed in the methods disclosed herein. Thus, the nucleotide constructs encompass all nucleotide constructs which can be employed in the methods for transforming maize plants including, but not limited to, those comprised of deoxyribonucleotides, ribonucleotides and combinations thereof. Such deoxyribonucleotides and ribonucleotides include both naturally occurring molecules and synthetic analogues. The nucleotide constructs also encompass all forms of nucleotide constructs including, but not limited to, single-stranded forms, double-stranded forms, hairpins, stem-and-loop structures and the like.

Furthermore, it is recognized that the methods may employ a nucleotide construct that is capable of directing, in a transformed plant, the expression of at least one protein, or at least one RNA, such as, for example, an rRNA, a tRNA and an antisense RNA that is complementary to at least a portion of an mRNA. Typically such a nucleotide construct is comprised of a coding sequence for a protein or an RNA operably linked to 5′ and 3′ transcriptional regulatory regions. Alternatively, it is also recognized that the methods may employ a nucleotide construct that is not capable of directing, in a transformed plant, the expression of a protein or an RNA.

In addition, it is recognized that methods do not depend on the incorporation of the entire nucleotide construct into the genome, only that the genome of the maize plant is altered as a result of the introduction of the nucleotide construct into a maize cell. Alterations to the genome include additions, deletions and substitution of nucleotides in the genome. While the methods do not depend on additions, deletions, or substitutions of any particular number of nucleotides, it is recognized that such additions, deletions or substitutions comprise at least one nucleotide.

The nucleotide constructs also encompass nucleotide constructs, that may be employed in methods for altering or mutating a genomic nucleotide sequence in an organism, including, but not limited to, chimeric vectors, chimeric mutational vectors, chimeric repair vectors, mixed-duplex oligonucleotides, self-complementary chimeric oligonucleotides and recombinogenic oligonucleobases.

The nucleotide constructs may comprise at least one expression cassette for expression in the maize plant of interest. The expression cassette can include 5′ and 3′ regulatory sequences operably linked to a gene of interest sequence of the invention. Operably linked indicates a functional linkage between two nucleotide sequences, for example a functional linkage of a promoter and a second sequence, such that the promoter sequence initiates and mediates transcription of the DNA sequence corresponding to the second sequence. In some examples, operably linked means that the nucleic acid sequences being linked are contiguous and, where necessary to join two protein coding regions, contiguous and in the same reading frame. The cassette may additionally contain at least one additional gene to be cotransformed into the organism. Alternatively, the additional gene(s) can be provided on multiple expression cassettes.

An expression cassette may be provided with a plurality of restriction sites for insertion of the gene of interest sequence to be under the transcriptional regulation of the regulatory regions. The expression cassette may additionally contain identification or selectable marker genes.

The expression cassette may include in the 5′-3′ direction of transcription, a transcriptional and translational initiation region, a gene of interest sequence of the invention, and a transcriptional and translational termination region functional in plants. The transcriptional initiation region, the promoter, may be native or analogous or foreign or heterologous to the plant host. Additionally, the promoter may be the natural sequence or alternatively a synthetic sequence. A “foreign” sequence is one that is not naturally found in the host plant, for example a foreign transcriptional initiation region is not found in the native plant into which the transcriptional initiation region is introduced. As used herein, a chimeric gene comprises a coding sequence operably linked to a transcription initiation region that is heterologous to the coding sequence.

In other examples, constructs which express the gene of interest using native promoter sequences may be used. Such constructs typically change expression levels of the gene of the interest in the plant or plant cell. Thus, it is expected that the phenotype of the plant or plant cell is altered.

The termination region may be native with the transcriptional initiation region, may be native with the operably linked DNA sequence of interest, or may be derived from another source. Convenient termination regions are available from the Ti-plasmid of A. tumefaciens, such as the octopine synthase and nopaline synthase termination regions. See also Guerineau et al. (1991) Mol Gen Genet. 262:141-144; Proudfoot (1991) Cell 64:671-674; Sanfacon et al. (1991) Genes Dev 5:141-149; Mogen et al. (1990) Plant Cell 2:1261-1272; Munroe et al. (1990) Gene 91:151-158; Ballas et al. (1989) Nucl Acids Res 17:7891-7903; and Joshi et al. (1987) Nucl Acid Res 15:9627-9639.

Where appropriate, the nucleotide sequence of interest, such as a gene(s) may be optimized for increased expression in the transformed plant. That is, the genes can be synthesized using plant-preferred codons for improved expression. See, for example, Campbell and Gowri (1990) Plant Physiol 92:1-11 for a discussion of host-preferred codon usage. Methods are available in the art for synthesizing plant-preferred genes. See, for example, U.S. Pat. Nos. 5,380,831, and 5,436,391 and Murray et at (1989) Nucl Acids Res 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′-leader sequences in the expression cassette construct. Such leader sequences can act to enhance translation. Translation leaders are known in the art and include: picornavirus leaders, for example, EMCV leader (Encephalomyocarditis 5′-noncoding region) (Elroy-Stein et al. (1989) Proc Natl Acad Sci USA 86:6126-6130); potyvirus leaders, for example, TEV leader (Tobacco Etch Virus) (Allison et al. (1986); MDMV leader (Maize Dwarf Mosaic Virus); Virology 154:9-20), and human immunoglobulin heavy-chain binding protein (BiP), (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol 84:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, recombination sites, removal of superfluous DNA, removal of restriction sites, operable linkages, fusions, or the like. For this purpose, in vitro mutagenesis, primer repair, restriction, annealing, resubstitutions, e.g., transitions and transversions, may be involved.

Any promoter can be used, and is typically selected based on the desired outcome. A promoter is a region of DNA involved in recognition and binding of RNA polymerase and other proteins to initiate transcription. A plant promoter is a promoter capable of initiating transcription in a plant cell, for a review of plant promoters see Potenza et al. (2004) In Vitro Cell Dev Biol 40:1-22. The nucleic acids can be combined with constitutive, tissue-preferred, developmental, inducible, or other promoters for expression in maize plants.

Depending on the desired result, it may be beneficial to express a gene under the control of an inducible promoter, particularly from a pathogen-inducible promoter. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth J Plant Pathol 89:245-254; Uknes et al. (1992) Plant Cell 4:645-656; and Van Loon (1985) Plant Mol. Virol. 4:111-116. See also WO99/43819, herein incorporated by reference.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc Natl Acad Sci USA 88:10421-10425 and McNellis et al. (1998) Plant J 14:247-257) and tetracycline-inducible and tetracycline-repressible promoters (see, for example, Gatz et al. (1991) Mol Gen Genet 227:229-237, and U.S. Pat. Nos. 5,814,618 and 5,789,156), herein incorporated by reference.

Various changes in phenotype are of interest including modifying the fatty acid composition in a plant, altering the amino acid content of a plant, altering a pathogen defense mechanism, modifying stress response, modifying yield, modifying nutrient needs and/or utilization, modifying plant architecture, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes or nucleotide sequences of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increases, the choice of genes for transformation will change accordingly. More specific categories of transgenes, for example, include genes encoding important traits for agronomics, insect resistance, disease resistance, herbicide resistance, sterility, grain characteristics, and commercial products. Genes of interest include, generally, those involved in oil, starch, carbohydrate, or nutrient metabolism as well as those affecting kernel size, sucrose loading, and the like.

Grain traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch.

Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,737,514; 5,723,756; 5,593,881; and Geiser et al. (1986) Gene 48:109); lectins (Van Damme et al. (1994) Plant Mol Biol 24:825); and the like.

Genes encoding disease resistance traits include detoxification genes, such as against fumonosin (see, e.g., U.S. Pat. Nos. 5,716,820, 5,792,931, 6,025,188, 6,229,071, and 6,573,075); avirulence (avr) and disease resistance (R) genes (Jones et al. (1994) Science 266:789; Martin et al. (1993) Science 262:1432; and Mindrinos et al. (1994) Cell 78:1089); and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene), or other such genes known in the art. The bar gene encodes resistance to the herbicide basta, the nptII gene encodes resistance to the antibiotics kanamycin and geneticin, and the ALS-gene mutants encode resistance to the herbicide chlorsulfuron.

Sterility genes can also be encoded in an expression cassette and provide an alternative to physical emasculation. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

The quality of seed is reflected in traits such as levels and types of oils, saturated and unsaturated, quality and quantity of essential amino acids, and levels of cellulose. For example, U.S. Pat. Nos. 5,990,389; 5,885,801; and 5,885,802 and U.S. Pat. No. 5,703,409, provide descriptions of modifications of proteins for desired purposes.

Commercial traits can also be encoded on a gene or genes that could increase for example, starch for ethanol production, or provide expression of proteins. Another important commercial use of transformed plants is the production of polymers and bioplastics such as described in U.S. Pat. No. 5,602,321.

Exogenous products include plant enzymes and products as well as those from other sources including prokaryotes and other eukaryotes. Such products include enzymes, cofactors, hormones, and the like. The level of proteins, particularly modified proteins having improved amino acid distribution to improve the nutrient value of the plant, can be increased.

Putative events, regenerated plants, and/or progeny thereof are usually subjected to various analyses to develop a molecular characterization of the event. Analyses include but are not limited to methods and tools that verify that the expression cassette(s) were transferred intact with no partial deletions, duplications, or rearrangement of elements, that detect the presence or absence of vector backbone, that measure the copy number of the transgene(s) of interest, and the like. Typically, events having a single copy of the transgene(s) of interest are selected for further analysis and/or advancement. Transformation experiments vary in the frequency of single copy events. For example, the frequency of single copy events can range from 10%-100% of the events, including but not limited to about 10%, 15%, 20%, 25%, 30%, 40%, 45%, 50%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 90%, 95%, or 100% of the total number of events generated. This invention can be better understood by reference to the following non-limiting examples. It will be appreciated by those skilled in the art that other embodiments of the invention may be practiced without departing from the spirit and the scope of the invention as herein disclosed and claimed.

### EXAMPLES

The present invention is further illustrated in the following Examples, in which parts and percentages are by weight and degrees are Celsius, unless otherwise stated. It should be understood that these examples, while indicating embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Furthermore, various modifications of the invention in addition to those shown and described herein will be apparent to those skilled in the art from the foregoing description. Such modifications are also intended to fall within the scope of the appended claims.

### Example 1

### Standard Media

Any transformation method and standard media can be used. The following is an exemplary set of media and protocols.

TABLE 2
Composition of media used:

| Media | Composition (Unit Volume = 1 L) |
|---|---|
| 561Q | 4.0 g Chu(N6) basal salts, 1 mL Eriksson's vitamins 1000X, 0.5 mg thiamine |
| | HCl, 1.5 mg 2,4-D, 0.69 g L-proline, 68.5 g sucrose, 36 g glucose, pH 5.2 |
| 562P | 4.0 g Chu(N6) basal salts, 1 mL Eriksson's vitamins 1000X, 0.5 mg thiamine |
| | HCl, 2.0 mg 2,4-D, 0.69 g L-proline, 30 g sucrose, 0.85 mg silver nitrate, 1 mL |
| | acetosyringone at 100 mM, 3.0 g Gelrite, pH 5.8 |
| 563O | 4.0 g Chu(N6) basal salts, 1 mL Eriksson's vitamins 1000X, 0.5 mg thiamine |
| | HCl, 1.5 mg 2,4-D, 0.69 g L-proline, 30 g sucrose, 0.5 g MES buffer, 0.85 mg |
| | silver nitrate, 3 mg Bialaphos, 100 mg carbenicillin, 8.0 g agar, pH 5.8 |
| 289B | 4.3 g MS basal salt mixture, 1 g myo-inositol, 0.5 mg nicotinic acid, 0.1 mg |
| | thiamine•HCl, 0.5 mg pyridoxine•HCl, 2 mg glycine, 0.5 mg zeatin, 1 mg IAA |
| | at 0.5 mg/mL, 1 mL ABA at 0.1 mM, 1.5 mg Bialophos, 100 mg carbenicillin, |
| | 60.0 g sucrose, 3.0 g Gelrite, pH 5.6. |
| 271C | 4.3 g MS basal salt mixture, 0.1 g myo-inositol, 0.5 mg nicotinic acid, 0.1 mg |
| | thiamine HCl, 0.5 mg pyridoxine HCl, 2 mg glycine, 40 g sucrose, 3 mg |
| | Bialaphos, 1.5 g Gelrite, pH 5.6. |
| 272 | 4.3 g MS basal salt mixture, 0.1 g myo-inositol, 0.5 mg nicotinic acid, 1 mg |
| | thiamine HCl, 0.5 mg pyridoxine•HCl, 2 mg glycine, 40 g sucrose, 1.5 g |
| | Gelrite, pH 5.6. |
| 800 | 3 g potassium phosphate dibasic, 1 g sodium phosphate monobasic anhydrous, |
| | 1 g ammonium chloride, 0.3 g magnesium sulfate heptahydrate, 0.15 g |
| | potassium chloride, 100 mg calcium chloride anhydrate, 25 mg ferrous sulfate |
| | heptahydrate, 9 g agar, 5 g glucose, 100 mg spectinomycin, pH 7.0 |
| 810D | 5 g yeast extract, 10 g Peptone, 5 g sodium chloride, 15 g agar, 50 mg |
| | spectinomycin, pH 6.8. |
| 12S | 5 g glucose, 15 g agar, 2.5 mg ferrous sulfate heptahydrate, 3 g potassium |
| | phosphate dibasic, 1 g sodium phosphate monobasic anhydrous, 1 g ammonium |
| | chloride, 0.3 g magnesium sulfate heptahydrate, 0.15 g potassium chloride, |
| | 14.4 mg calcium chloride anhydrate, 50 mg spectinomycin. |

### Example 2

### One Embodiment of an Agrobacterium Transformation Protocol of GS3 & GS3XGaspe with PAT Selection that Typically Uses 6 Steps and Lasts about 11 Weeks to Obtain T₀ Plants from Embryos

### Isolation of Fresh Embryos

1. Ears are harvested when the embryo size reaches 1.0-2.0 mm. The ear source are from GH (Johnston Greenhouse) or SH (Johnston an open field covered by screen) or GC (growth chamber of Conviron-BDW120).
2. Sterilize the ears with a 20%-30% bleach solution made with diH₂O adding 2-4 drops of Tween 20, for 20 minutes (no longer than 30 minutes). Drain the solution from each container and rinse three times with sterile diH₂O
3. Add 2 mL of 561Q medium into a sterile 2 mL microcentrifuge tube for embryo isolation. Label the tops and sides of the microcentrifuge tubes if needed.
4. Dissect embryos from an ear and drop them into a microcentrifuge tube containing 561Q.

### Preparation of Agrobacterium Suspension for Agroinfection

5. Agrobacterium master plate: Pick up frozen Agrobacterium (−80° C.) and streak on 800 or 12S medium and culture at 28° C. in dark for 2-3 days. This plate can be stored at 4° C. and used usually for 1 month.
6. Pick up a colony from the master plate and streak on an 810D medium plate (containing 50 mg/L Spectinomycin) and incubate in the dark at 28° C. for 1-2 days.
7. Collect the Agrobacterium growth from this plate with a loop and suspend it into 14 mL Falcon tube with 561Q medium and shake by hand to reach an even suspension.
8. Take 1 mL of the solution and dispense into a disposable spectrophotometer cuvette and use 561Q as control. Adjust the suspension to give an OD of 0.35-0.45 at 550 nm under visible light. Agrobacterium concentration is 1×10⁹ cfu/mL at an OD of 0.72.

### Agrobacterium Infection of Embryos, Co-Culture

9. Remove the medium from the tube containing the fresh embryos.
10. Add 1 mL of the Agrobacterium suspension at OD described above and vortex at low speed for 15-30 second.
11. Stand the tube for 5 minutes at room temperature in the hood.
12. Pour the suspension with embryos onto 562P plate. Transfer any embryos that are left in the tube or cap onto the plate with a sterile spatula. Check that the plate is labeled to include: Agro ID (option: ear source, ear genotype, ear number, pollination and harvest dates).
13. Remove the extra Agrobacterium with a pipette, and place the embryos axis down on the medium. All of the embryos from a single ear are placed on one 562P plate. Seal the plate with Para film.
14. Incubate the plate in the dark for 3 days at 21° C.
15. Transfer the plate in dark for 4-7 days at 26° C.

### Selection and Regeneration

16. Transfer all of the embryos from 562P to the plate containing 563O medium. Spread out about 20 embryos per plate (the best time to count embryo number). Seal the plate with Para film. Incubate the plates in the dark at 26° C.
17. After two weeks, subculture the embryos onto 563O and continue incubation under the same conditions. Seal the plate with Para film.
18. After four weeks, pick up events based on one event per embryo. Place one event to single 289B plate as a small amount in a one spot with the label indicated as early event. Incubate all events in 289B in the dark for two weeks at 26° C. for the conversion of immature somatic embryos into matured somatic embryos.

No more than 24 constructs per week enter into regeneration medium 289B.

Record event numbers, and total embryos number in common frequency sheet.
19. After two weeks, each event that has visible shoots and roots is transferred onto each Phytotray with 271C or 272 medium and is placed under artificial light at 26° C.

Record event number with any green leave in common frequency sheet.
20. After two weeks, send 10 Phytotrays with most vigorous plants to Greenhouse. Record event in Datagrid of Greenhouse icon.

### Example 3

### One Embodiment of an Agrobacterium Transformation Protocol of GS3 & GS3XGaspe with PAT Selection that Typically Uses 8 Steps and Lasts about 12 Weeks

### Isolation of Fresh Embryos

1. Ears are harvested when the embryo size reaches 1.0-2.0 mm. The ear source are from GH (Johnston Greenhouse) or SH (Johnston an open field covered by screen) or GC (growth chamber of Conviron-BDW120).
2. Sterilize the ears with a 20%-30% bleach solution made with diH₂O adding 2-4 drops of Tween 20, for 20 minutes (no longer than 30 minutes). Drain the solution from each container and rinse three times with sterile diH₂O
3. Add 2 mL of 561Q medium into a sterile 2 mL microcentrifuge tube for embryo isolation. Label the tops and sides of the microcentrifuge tubes if needed.
4. Dissect embryos from an ear and drop them into a microcentrifuge tube containing 561Q.

### Preparation of Agrobacterium Suspension for Agroinfection

5. Agrobacterium master plate: Pick up frozen Agrobacterium (−80° C.) and streak on 800 or 12S medium and culture at 28° C. in dark for 2-3 days. This plate can be stored at 4° C. and used usually for 1 month.
6. Pick up a colony from the master plate and streak on an 810D medium plate (containing 50 mg/L Spectinomycin) and incubate in the dark at 28° C. for 1-2 days.
7. Collect the Agrobacterium growth from this plate with a loop and suspend it into 14 mL Falcon tube with 561Q medium and shake by hand to reach an even suspension.
8. Take 1 mL of the solution and dispense into a disposable spectrophotometer cuvette and use 561Q as control. Adjust the suspension to give an OD of 0.35-0.45 at 550 nm under visible light. Agrobacterium concentration is 1×10⁹ cfu/mL at an OD of 0.72.

### Agrobacterium Infection of Embryos, Co-Culture

9. Remove the medium from the tube containing the fresh embryos.
10. Add 1 mL of the Agrobacterium suspension at OD described above and vortex at low speed for 15-30 second.
11. Stand the tube for 5 minutes at room temperature in the hood.
12. Pour the suspension with embryos onto 562P plate. Transfer any embryos that are left in the tube or cap onto the plate with a sterile spatula. Check that the plate is labeled to include: Agro ID (option: ear source, ear genotype, ear number, pollination and harvest dates).
13. Remove the extra Agrobacterium with a pipette, and place the embryos axis down on the medium. All of the embryos from a single ear are placed on one 562P plate. Seal the plate with Para film.
14. Incubate the plate in the dark for 3 days at 21° C.
15. Transfer the plate in dark for 4-7 days at 26° C.

### Selection and Regeneration

16. Transfer all of the embryos from 562P to the plate containing 563O medium. Spread out about 20 embryos per plate (the best time to count embryo number). Seal the plate with Para film. Incubate the plates in the dark at 26° C.
17. After two weeks, subculture the embryos onto 563O and continue incubation under the same conditions. Seal the plate with Para film.
18. After three weeks, pick up the events based on one event per embryo. Place one event to single 563O plate with the label indicated as early event. If less than 12 events picked or events with bad quality, transfer the rest of embryos to fresh 563O plate.
19. After two weeks, find more events indicated as later event if less than 12 events picked or events with bad quality. Transfer the both early and later apparent embryogenic events to 289B medium as a small amount in a one spot. Incubate all events in 289B in the dark for two weeks at 26° C. for the conversion of immature somatic embryos into matured somatic embryos.

No more than 24 constructs per week enter into regeneration medium 289B.

Record early and later event numbers, and total embryos number in common frequency sheet.
20. After two weeks, the material that has visible shoots and roots is transferred onto 271C or 272 medium and is placed under artificial light at 26° C.
21. One week later the plantlets are placed into tubes containing 272 mediums. Generally two plantlets per event.

Record event number with any green leave in common frequency sheet.
22. Choose one healthiest, most vigorous plant per event, 10 plants from 10 events and send them to Greenhouse. Record early or later event in Datagrid of Greenhouse icon.

### Example 4

### One Embodiment of an Agrobacterium Transformation Protocol of GS3XGaspe with GAT Selection that Typically Uses 6 Steps and Lasts about 11 Weeks to Obtain T₀ Plants from Embryos

### Isolation of Fresh Embryos

1. Ears are harvested when the embryo size reaches 1.0-2.0 mm. The ear source are from GH (Johnston Greenhouse) or SH (Johnston an open field covered by screen) or GC (growth chamber of Conviron-BDW120).
2. Sterilize the ears with a 20%-30% bleach solution made with diH₂O adding 2-4 drops of Tween 20, for 20 minutes (no longer than 30 minutes). Drain the solution from each container and rinse three times with sterile diH₂O
3. Add 2 mL of 561Q medium into a sterile 2 mL microcentrifuge tube for embryo isolation. Label the tops and sides of the microcentrifuge tubes if needed.
4. Dissect embryos from an ear and drop them into a microcentrifuge tube containing 561Q.

### Preparation of Agrobacterium Suspension for Agroinfection

5. Agrobacterium master plate: Pick up frozen Agrobacterium (−80° C.) and streak on 800 or 12S medium and culture at 28° C. in dark for 2-3 days. This plate can be stored at 4° C. and used usually for 1 month.
6. Pick up a colony from the master plate and streak on an 810D medium plate (containing 50 mg/L Spectinomycin) and incubate in the dark at 28° C. for 1-2 days.
7. Collect the Agrobacterium growth from this plate with a loop and suspend it into 14 mL Falcon tube with 561Q medium and shake by hand to reach an even suspension.
8. Take 1 mL of the solution and dispense into a disposable spectrophotometer cuvette and use 561Q as control. Adjust the suspension to give an OD of 0.35-0.45 at 550 nm under visible light. Agrobacterium concentration is 1×10⁹ cfu/mL at an OD of 0.72.

### Agrobacterium Infection of Embryos, Co-Culture

9. Remove the medium from the tube containing the fresh embryos.
10. Add 1 mL of the Agrobacterium suspension at OD described above and vortex at low speed for 15-30 second.
11. Stand the tube for 5 minutes at room temperature in the hood.
12. Pour the suspension with embryos onto 562P plate. Transfer any embryos that are left in the tube or cap onto the plate with a sterile spatula. Check that the plate is labeled to include: Agro ID (option: ear source, ear genotype, ear number, pollination and harvest dates).
13. Remove the extra Agrobacterium with a pipette, and place the embryos axis down on the medium. All of the embryos from a single ear are placed on one 562P plate. Seal the plate with Para film.

Record Co-cultivation date in common TXN Tracking sheet every week.
14. Incubate the plate in the dark for 3 days at 21° C.
15. Transfer the plate in dark for 4-7 days at 26° C.

### Selection and Regeneration

16. Transfer all of the embryos from 562P to the plate containing 563V medium. Spread out about 20 embryos per plate (the best time to count embryo number). Seal the plate with Para film (optional). Incubate the plates in the dark at 26° C.
17. After two weeks, subculture the embryos onto 563M and continue incubation under the same conditions. Seal the plate with Para film (optional).
18. After four weeks, pick up events based on one event per embryo. Place one event to single 287M plate as a small amount in a one spot with the label indicated as early event. Incubate all events in 289B in the dark for two weeks at 26° C. for the conversion of immature somatic embryos into matured somatic embryos.

No more than 24 constructs per week enter into regeneration medium 287M.

Record event numbers, and total embryos number in common frequency sheet.
19. After two weeks, each event that has visible shoots and roots is transferred onto each Phytotray with 273I or 272 medium and is placed under artificial light at 26° C.

Record event number with any green leave in common frequency sheet.
20. After two weeks, send 10 Phytotrays with most vigorous plants to Greenhouse. Record event in Datagrid of Greenhouse icon.

### Example 5

### One Embodiment of an Agrobacterium Transformation Protocol of GS3XGaspe with GAT Selection that Typically Uses 8 Steps and Lasts about 12 Weeks to Obtain T₀ Plants from Embryos

### Isolation of Fresh Embryos

1. Ears are harvested when the embryo size reaches 1.0-2.0 mm. The ear source are from GH (Johnston Greenhouse) or SH (Johnston an open field covered by screen) or GC (growth chamber of Conviron-BDW120).
2. Sterilize the ears with a 20%-30% bleach solution made with diH₂O adding 2-4 drops of Tween 20, for 20 minutes (no longer than 30 minutes). Drain the solution from each container and rinse three times with sterile diH₂O
3. Add 2 mL of 561Q medium into a sterile 2 mL microcentrifuge tube for embryo isolation. Label the tops and sides of the microcentrifuge tubes if needed.
4. Dissect embryos from an ear and drop them into a microcentrifuge tube containing 561Q.

Preparation of Agrobacterium Suspension for Agroinfection
5. Agrobacterium master plate: Pick up frozen Agrobacterium (−80° C.) and streak on 800 or 12S medium and culture at 28° C. in dark for 2-3 days. This plate can be stored at 4° C. and used usually for 1 month.
6. Pick up a colony from the master plate and streak on an 810D medium plate (containing 50 mg/L Spectinomycin) and incubate in the dark at 28° C. for 1-2 days.
7. Collect the Agrobacterium growth from this plate with a loop and suspend it into 14 mL Falcon tube with 561Q medium and shake by hand to reach an even suspension.
8. Take 1 mL of the solution and dispense into a disposable spectrophotometer cuvette and use 561Q as control. Adjust the suspension to give an OD of 0.35-0.45 at 550 nm under visible light. Agrobacterium concentration is 1×10⁹ cfu/mL at an OD of 0.72.

Agrobacterium Infection of Embryos, Co-Culture
9. Remove the medium from the tube containing the fresh embryos.
10. Add 1 mL of the Agrobacterium suspension at OD described above and vortex at low speed for 15-30 second.
11. Stand the tube for 5 minutes at room temperature in the hood.
12. Pour the suspension with embryos onto 562P plate. Transfer any embryos that are left in the tube or cap onto the plate with a sterile spatula. Check that the plate is labeled to include: Agro ID (option: ear source, ear genotype, ear number, pollination and harvest dates).
13. Remove the extra Agrobacterium with a pipette, and place the embryos axis down on the medium. All of the embryos from a single ear are placed on one 562P plate. Seal the plate with Para film.

Record Co-cultivation date in common TXN Tracking sheet every week.
14. Incubate the plate in the dark for 3 days at 21° C.
15. Transfer the plate in dark for 4-7 days at 26° C.

Selection and Regeneration
16. Transfer all of the embryos from 562P to the plate containing 563V medium. Spread out about 20 embryos per plate (the best time to count embryo number). Seal the plate with Para film (optional). Incubate the plates in the dark at 26° C.
17. After two weeks, subculture the embryos onto 563M and continue incubation under the same conditions. Seal the plate with Para film (optional).
18. After three weeks, pick up the events based on one event per embryo. Place one event to single 563M plate with the label indicated as early event. If less than 12 events picked or events with bad quality, transfer the rest of embryos to fresh 563M plate.
19. After two weeks, find more events indicated as later event if less than 12 events picked or events with bad quality. Transfer the both early and later apparent embryogenic events to 287M medium as a small amount in a one spot. Incubate all events in 287M in the dark for two weeks at 26° C. for the conversion of immature somatic embryos into matured somatic embryos.

No more than 24 constructs per week enter into regeneration medium 287M.

Record early and later event numbers, and total embryos number in common frequency sheet.
20. After two weeks, the material that has visible shoots and roots is transferred onto 273I medium and is placed under artificial light at 26° C.
21. One week later the plantlets are placed into tubes containing 273I mediums. Generally two plantlets per event.

Record event number with any green leave in common frequency sheet.
22. Choose one healthiest, most vigorous plant per event, 10 plants from 10 events and send them to Greenhouse. Record early or later event in Datagrid of Greenhouse icon.

The article “a” and “an” are used herein to refer to one or more than one (i.e., to at least one) of the grammatical object of the article. By way of example, “an element” means one or more element.

All publications and patent applications in this specification are indicative of the level of ordinary skill in the art to which this invention pertains. All publications and patent applications are herein incorporated by reference to the same extent as if each individual publication or patent application was specifically and individually indicated by reference.

The invention has been described with reference to various specific and preferred embodiments and techniques. However, it should be understood that many variations and modifications may be made while remaining within the spirit and scope of the invention.

What is claimed is:

1. A method for producing a transformed maize plantlet, said method comprising:
(a) isolating an immature embryo from a maize ear;
(b) introducing a polynucleotide of interest into at least one cell of said immature embryo to produce a transformed maize cell;
(c) placing said immature embryo in or on selection medium to identify transgenic calli, wherein no more than 2 rounds of selection are performed;
(d) culturing the transgenic calli comprising immature somatic embryos in or on a maturation medium to produce mature somatic embryos; and
(e) regenerating a transformed maize plantlet from the mature somatic embryo in a plant cell culture vessel that allows for root formation and plantlet elongation in the same vessel, and wherein the transformed maize plantlet comprises the polynucleotide of interest.

2. The method of claim 1, wherein the immature embryo is subjected to selection medium for about 6 weeks or less.

3. The method of claim 1, comprising selecting for the transformed maize cells in or on selection medium for about 2 weeks and then subculturing the transformed maize cells in or on selection medium for about 4 weeks.

4. The method of claim 1, comprising placing the immature embryo in or on selection medium until the transgenic calli are formed.

5. The method of claim 1, wherein the immature embryos are placed in or on selection medium for two rounds of selection.

6. The method of claim 1, wherein the plant cell culture vessel is not a petri dish or a test tube.

7. The method of claim 1, wherein the plant cell culture vessel is capable of holding a plurality of plantlets.

8. The method of claim 1, comprising placing a plurality of mature somatic embryos in the plant cell culture vessel to regenerate a plurality of transgenic maize plantlets.

9. The method of claim 1, wherein the plant cell culture vessel is a phytotray plant cell culture vessel.

10. The method of claim 1, wherein the mature somatic embryos are transferred to a plant cell culture vessel comprising regeneration medium.

11. The method of claim 1, wherein the mature somatic embryos are transferred to a plant cell culture vessel comprising regeneration medium for a period of about 2 weeks.

12. The method of claim 1, comprising transferring the plantlets into pots and growing the plantlets into plants.

13. The method of claim 1, wherein the polynucleotide of interest is introduced using Agrobacterium, particle bombardment, electroporation, PEG-induced transfection, particle bombardment, silicon fiber delivery or microinjection.

14. The method of claim 1, wherein the method decreases the period of time from introducing the polynucleotide of interest and regenerating the transgenic plantlet as compared to a control method by at least one week.
